(19) United States
(12) Patent Application Publication
(10) Pub. No.: US 2025/0064377 A1
(43) Pub. Date: Feb. 27, 2025

(54) METHOD FOR ASCERTAINING A SETPOINT VALUE AND FOR REPRESENTING MEDICAL IMAGE DATA, PROCESSING APPARATUS, COMPUTER PROGRAM AND DATA CARRIER

(71) Applicant: Siemens Healthineers AG, Forchheim (DE)

(72) Inventor: Alois Regensburger, Poxdorf (DE)

(21) Appl. No.: 18/812,328

(22) Filed: Aug. 22, 2024

(30) Foreign Application Priority Data
Aug. 24, 2023 (DE) 10 2023 208 106.7

(51) Int. Cl. A61B 5/00; G06T 7/00; G06T 7/10; G16H 30/20; G16H 30/40

(52) U.S. Cl. CPC A61B 5/4064; A61B 5/0042; A61B 5/7267; G06T 7/0012; G06T 7/10; G16H 30/20; G16H 30/40; G06T 2207/30016

(57) ABSTRACT

Systems and methods for ascertaining a setpoint value for at least one control parameter that serves to control an acquisition and/or processing and/or a representation of medical image data. At least one measurement dataset is obtained that is based on an acquisition by way of sensors of a brain activity of a reference person while the reference person observes at least one representation, wherein the representation is based on a medical image dataset. The setpoint value is ascertained as a function of the measurement dataset.

### CROSS REFERENCE TO RELATED APPLICATIONS

This application claims the benefit DE 10 2023 208 106.7 filed on Aug. 24, 2023, which is hereby incorporated by reference in its entirety.

### FIELD

Embodiments relate to a computer-implemented method for ascertaining a respective setpoint value for at least one control parameter which serves to control an acquisition and/or processing and/or a representation of medical image data.

### BACKGROUND

In the field of medical imaging, image datasets are frequently visualized for an observer who uses this visualization, for example for diagnosis purposes or for monitoring a medical intervention. The observer should therefore be provided with an image that is as easy to interpret as possible. This aim is often at odds with other demands on the imaging, however, for example for limiting an irradiated X-ray intensity and/or for achieving a high imaging rate or a short measuring time.

It is therefore known per se to adjust parameters of the acquisition and/or processing and/or representation of medical image data, in order to optimize the image quality, during the course of the production and configuration of imaging medical examination facilities and sometimes also dynamically during measuring operation.

Quality measures that may be automatically ascertained may be adopted here, for example a minimization of a noise component and/or a contrast maximization. It has been found, however, that sometimes such optimizations do not result in an image impression that actually makes optimally easy and robust identification of image contents possible for the observer.

Studies may be carried out as to how different image impressions affect observers, for example in that a selected group of observers, for example of doctors and/or other medical experts, are shown different representations of medical image data in each case that are based on a different parameterization. The subjective image impressions may then be acquired and statistically evaluated by way of questionnaires or the like.

In corresponding studies, screened representations are sometimes evaluated with a tendency towards visual preference, however, so, for example as a result of the resulting parameterization, there is a risk of suppressing features in the representation that are actually present if this results in a clearer image impression or the like at first glance. In addition, corresponding studies may take account of the fact that differently parameterized representations may be advantageous for different observers to only a very limited extent.

### BRIEF SUMMARY AND DESCRIPTION

The scope of the embodiments is defined solely by the appended claims and is not affected to any degree by the statements within this summary. The present embodiments may obviate one or more of the drawbacks or limitations in the related art. Independent of the grammatical term usage, individuals with male, female or other gender identities are included within the term.

Embodiments improve the acquisition, processing and representation of medical image data.

Embodiments provide a method that includes the following steps: obtaining at least one measurement dataset that is based on an acquisition by way of sensors of a respective brain activity of a respective reference person, while the reference person observes at least one representation, wherein the respective representation is based on a respective medical image dataset, and ascertaining the setpoint value as a function of the measurement dataset.

By taking into account the brain activity of at least one reference person in the parameterization of the procedures, it is possible to optimize the visualization of the medical image data, that takes into account the actual human perception of the at least one reference person and/or the medical image data may be enhanced by way of semantic content that was identified, for example, by the reference person. Different approaches to this will be explained in more detail below.

During the course of research on computer-brain interfaces it has been found that images observed by an observer may be at least approximately reconstructed on the basis of instances of brain activity acquired by sensors. Suitable measurement datasets may be or are ascertained, for example, by a functional magnetic resonance imaging of the brain and/or by measuring brain waves (electroencephalography) and/or by chips implanted in the brain or interfaces.

Corresponding methods are discussed, for example, in the articles Yu Takagi et al., High-resolution image reconstruction with latent diffusion models from human brain activity, bioRxiv 2022.11.18.517004; doi: https://doi.org/10.1101/2022.11.18.517004, and Zijiao Chen et al., Seeing Beyond the Brain: Conditional Diffusion Model with Sparse Masked Modeling for Vision Decoding, arXiv preprint arXiv:2211.06956 (2022), https://arxiv.org/abs/2211.06956. Here algorithms trained by machine learning in each case are used in order to reconstruct the originally observed image from measurement data of a functional magnetic resonance imaging.

As is represented in detail in the article Yu Takagi et al., Improving visual image reconstruction from human brain activity using latent diffusion models via multiple decoded inputs, arXiv preprint arXiv:2306.11536 [q-bio.NC](2023), https://doi.org/]0.48550/arXiv.2306.11536, images generated directly on the basis of the brain activity are, as a rule, blurred, but the semantic content of the image, i.e. for example a keyword relating to the image content, or a header of the image content, may be robustly ascertained.

The semantic content may be ascertained directly from the measured brain activity or the measurement dataset, whereby, for example, the semantic identification of represented features may be taken into account by the reference person. In addition, or as an alternative, generated images, that are based, for example, on measurement data in respect of the visual cortex, may be taken into account when identifying the semantic content, however.

Ascertaining viewed image contents on the basis of a measured brain activity may also be used to parameterize the acquisition, processing and/or the representation of medical image data.

For example, the evaluation of the measurement dataset in the method may be used to check whether the respective reference person identifies one or more relevant feature(s) that are present in the respective representation, i.e. for example the presence of a microcatheter mapped in a fluoroscopy image. The information as to whether a particular feature is semantically correctly identified may therefore be used as an automatically ascertained quality measure for the representation or its parameterization in order to determine or optimize the control parameter(s). In addition, or as an alternative, this information may be evaluated in order to ascertain whether particular contents are present in the representation, if it may be assumed that the reference person would identify them.

If conventional classification algorithms, trained for example by machine learning, are used for ascertaining a semantic image content, a measure for the robustness or unambiguousness of the identification may also be ascertained here, so it may thus additionally or alternatively be taken into account, for example as a quality measure, for how well or how easily the respective reference person identifies or perceives the respective feature.

Thus, for example, at least one control parameter may be varied until identification of relevant features is ensured or in order to maximize the measure for the robustness or unambiguousness of the identification.

In addition, or as an alternative, a check as to whether a particular feature is identified or an evaluation of the measure for the robustness or unambiguousness of the identification may also be used to check whether the reference person directed their attention to the representation during the acquisition of measurement data. In other words, the quality measure may serve as a measure of attention or a measure of attention may be determined as a function of the quality measure and the target parameter or at least one of the target parameters may be determined as a function of the measure of attention.

This may be expedient, for example, when the measurement dataset is evaluated during the course of a continuous or repeated acquisition of medical image data, for example during the course of fluoroscopy, for example in order to reduce the X-ray dose if the reference person does not pay the respectively current representation any attention or pays it only little attention.

Alternatively, or in addition, for example strictly specified sets of reference values may be used for the control parameters, from which that set of target parameters may be selected or as a function of that set of target parameters it may be ascertained that which maximizes the robustness or unambiguousness of the identification for a particular reference person or, for example, the mean of the quality measure for a group of reference persons.

This may be used, for example, to optimize what is known as a “flavor” of the representation for an optimum identification of features or objects represented in the image, i.e. for example to adjust how much a noise should be suppressed and/or edges sharpened.

The described procedure may also be used, for example, to identify limits for control parameters. For example, during fluoroscopy it is possible to monitor whether a particular feature, for example a microcatheter, is identified by the reference person or how robust this identification is and the X-ray dose may be increased as a function of this if the identification is not robust or is not sufficiently robust.

It is thus possible to determine on the basis of the measurement dataset by way of the procedure a quality measure for the respective representation and thus for the control parameters forming the basis of this representation. In the same way, as is known for other quality measures, for example for a contrast or a noise component, the setpoint values may then be optimized for the control parameters in respect of the quality measure. Since this quality measure may be automatically ascertained such an optimization is also possible during ongoing operation, for example during fluoroscopy imaging. Since the actual perception of the reference person or reference persons is taken into account, however, the problems mentioned in the introduction, that may occur with exclusive use of conventional quality measures, are avoided.

Since specific measurement data may be used, falsifications owing to a subjective assessment of the image impression, as may occur with the group studies based on questionnaires or the like mentioned in the introduction, may be avoided, moreover.

In addition, or as an alternative to optimization of the image quality in respect of the identification of particular image features, the procedure may be used, for example, to enhance the medical image dataset or a result of processing, that results due to processing of the medical image dataset, with items of semantic information and/or to train an algorithm that may carry out such an enhancement.

For example, an item of semantic information ascertained on the basis of the measurement dataset, which information relates to the content of the representation viewed by the reference person, may be used for segmentation and/or for marking or for labeling particular image parts and/or for training an algorithm, that carries out a segmentation or a labeling.

Since segmentation or labeling is a processing of medical image data, the ascertained semantic information or, generally, an item of information ascertained using the semantic information or using the measurement dataset, that serves for segmentation and/or for labeling, or an ascertained parameterization of an algorithm suitable for this, forms a control parameter.

After the control parameter has been ascertained it may be used directly to control the acquisition and/or the processing and/or the representation of medical image data and/or it may be provided via an output interface, that is, for example, a hardware interface or a software interface, i.e. for example be transferred to an external facility or be stored in a database.

The measurement dataset may be ascertained during the course of the method or, for example, also already exist before the beginning of the method and be obtained via an input interface, that may be a hardware interface or a software interface. For example, the measurement dataset may be stored in a database before the beginning of the method and be retrieved from it in order to carry out the method.

Irrespective of whether the measurement dataset is acquired as part of the method or before the beginning of the method, the acquisition of the measurement dataset may include, for example, the following steps: outputting the representation of the at least one medical image dataset and/or the representation of a processing result of the respective medical image dataset to the at least one reference person, and acquisition by way of sensors of respective measurement data relating to the brain activity of the respective reference person while the respective reference person observes the respective representation.

The image dataset or one of the image datasets may include the medical image data to be processed or displayed, although this is not imperative. For example, the at least one control parameter may be ascertained on the basis of medical reference data that may be acquired in advance or may be ascertained, for example, by simulation. This is expedient, for example, if the acquisition of the medical image data is to be parameterized.

A plurality of representations may also be taken into account that are based on the same medical image dataset, with for example different control parameters being used for processing and/or for representing this image dataset. This may serve, for example, for the selection of an optimum set of control parameters from a plurality of parameter sets and/or for iterative optimizing of the at least one control parameter.

The respective representation may depend on a respective reference value associated with the representation for the respective control parameter, wherein the respective setpoint value for the respective control parameter may also be ascertained as a function of the reference value for the respective control parameter that is associated with the representation or at least one of the representations.

The respective representation may thus be regarded as an example of the effect of the respective reference value of the respective control parameter. The measurement data ascertained on the basis of this representation may thus be evaluated to ascertain to what extent the at least one reference value associated with the respective representation is suitable as the setpoint value. This may serve, for example, for selecting a suitable set of setpoint values for the control parameters from a plurality of sets of reference values or for adjusting, for example in an iterative method, a control parameter until a desired representation quality is achieved.

The respective representation, for example the acquisition of the medical image dataset and/or the processing of the medical image dataset for providing a processing result to be represented and/or representing the medical image dataset and/or the processing result, may depend on the respective reference value associated with the representation for the respective control parameter. For example, the acquisition and/or processing and/or representation of the respective medical image dataset or the processing result may occur in the same way as for the medical image data, with the respective control parameter being specified here by the respective reference value. Processing of the medical image data may differ from the generation of the representation for example solely by way of the values of the control parameters and/or by way of the object mapped by the medical image data or the medical image dataset.

On the basis of the respective measurement dataset or on the basis of a respective group of measurement datasets, that are based on an acquisition by way of sensors of the respective brain activity of different reference persons when observing the same representation, a respective quality measure may be ascertained for the respective representation, wherein the setpoint value or at least one of the setpoint values is ascertained as a function of the quality measure. Observation of the same representation is here taken to mean, for example, also the observation of representations that are generated in the same way with the same control parameters on the basis of the same measurement dataset, even if processing and/or representation takes place by way of mutually different apparatuses. The quality measure may indicate, for example, whether or how unambiguously and/or robustly the measured brain activity indicates the identification of a specified relevant feature of the respective representation. Examples of this have already been explained above.

The setpoint value for the at least one control parameter may thus be specified, for example, as a function of which quality measure is achieved when using the respective reference value for the respective control parameter, and the reference value may be used as a function of the quality measure as the setpoint value or the setpoint value may be ascertained by adjusting the reference value.

For some control parameters, for example for control parameters that relate to the acquisition of the medical image data, for example for an irradiated X-ray intensity and/or a measuring time used, it may be known in which direction the control parameter has to be changed in order to increase the quality measure. In this case, for example an offset may be determined for the reference value on the basis of the ascertained quality measure, which offset may be added to this value in order to determine the setpoint value. With repeated or continuous imaging, the quality measure may be regulated, for example, to a setpoint value or in a target interval.

In addition, or as an alternative, the quality measure may also be used, for example with repeated or continuous imaging, to decide whether the attention of the reference person is directed toward the representation. For example, a drop in the quality measure below a particular threshold value and/or a drop in the quality measure by a particular value or factor within a specified number of imaging cycles may indicate that the reference person is averting or has averted their attention from the representation. This may be used as a trigger, for example in the case of X-ray imaging, to adjust a measuring protocol, i.e. for example in order to reduce the irradiated X-ray intensity or even to terminate the imaging, whereby, for example, unnecessary exposure of the examination object and/or unnecessary energy consumption may be avoided.

For some control parameters, for example for the degree of an edge sharpening and/or a noise suppression and/or for selection of a filter kernel, the correlation between quality measure and value of the control parameter may be non-monotone or different control parameters may interact in a complex manner. In these cases, for example, the quality measure may be optimized by variations in the control parameter or the control parameters. This may occur by way of conventional optimization methods or also by way of a selection of the set of reference values of the representation with the highest quality measure as the set of setpoint values. For this purpose, measurement datasets may be evaluated for representations that are based on different reference values for the control parameters.

With repeated or continuous imaging this optimization may occur during ongoing operation. Alternatively, such an optimization may also occur offline in that differently parameterized representations may be accordingly output to a particular reference person or a group of reference persons in order to ascertain and evaluate a large number of measurement datasets. The target parameters ascertained during the course of the optimization of the quality measure may then be used for this reference person or, for example in the case of an evaluation of measurement data of a group of reference persons, also generally for all users or a particular group of users for acquisition, processing and/or visualization of the medical image data of a subsequent imaging.

The control parameter or at least one of the control parameters may serve to control the processing and/or representation of the medical image data, wherein on the basis of the respective medical image dataset in each case, a plurality of mutually different representations is generated in that a processing and representation of the respective medical image dataset occurs in the same way as for the medical image data, wherein for providing the respective representation a respective reference value associated with the representation is used for specifying the respective control parameter, wherein the mutually different representations differ from one another at least in respect of the reference value for at least one of the control parameters, wherein as a function of the quality measure ascertained for the respective representation one of the representations or a subgroup of the representations that does not incorporate all representations is selected, according to which the setpoint value is specified for the respective control parameter as a function of the selected representation or of the respective reference value associated with the respective selected representation.

For example, the representation with the highest quality value may be selected and the reference value associated with this control parameter in the selected representation may be used as the setpoint value for the respective control parameter. It may be more advantageous, however, for example when similar quality values result for a plurality of representations, to select the subgroup of representations with the best quality values and to ascertain the setpoint value for the respective control parameter, for example, by finding the mean or median of the reference values for this control parameter in the subgroup or to also carry out an interpolation between this reference value as a function of the quality measure.

The procedure may thus be used, for example after imaging has already been concluded, to optimize processing and/or representation of the medical image data in such a way that, for example with given image data, a particular reference person may acquire items of relevant information in the generated representation particularly easily or that on the basis of studies with a plurality of reference persons, it is probable that items of relevant information may be found particularly easily from the representation for all observers or for a particular group of observers.

For example, different sets of reference values may be specified from whose use different image impressions, for example a sharper or less sharp image, different contrasts and/or image brightnesses or the like, result. Since the selection of control parameters, that primarily influence the image impression, has until now primarily depended, as a rule, on the user's taste and is shaped to a certain extent by subjective preferences, the different possible image impressions or parameter sets are also referred to as the “flavor” of the representation. For example, parameters of an edge sharpness, noise suppression and/or an averaging over time may be used as control parameters that influence the “flavor” of the representation.

It has been found that the subjectively preferred image impression does not correspond in all cases to that parameterization of the representation in which identification of particular anatomic features or relevant objects is particularly easily and robustly possible for the user. It may therefore be expedient to automatically specify the “flavor” of the representation or to indicate to a user a particularly suitable “flavor” at least for them or a particular target group.

It may be expedient to determine the optimum image impression for in each case exactly one reference person, for example to specify a presetting for this reference person on a subsequent observation of the medical image data. However, in order to determine an image impression that, at least for the majority of users, enables good identification of relevant features or objects in the image, for example for a particular application, and may thus be used, for example, as a general presetting for processing and/or representing the medical image data, as explained above, instances of brain activity of different reference persons when observing the same representation may be evaluated in order to ascertain a quality measure that takes into account the perception of the representation by different reference persons.

The medical image dataset, on which the representation is based, may be a medical image dataset ascertained during the course of an imaging sequence, with the imaging sequence including repeated or continuous acquisition of medical image data on the same examination object, it being possible for the setpoint value to serve for control of the acquisition and/or processing and/or representation of at least parts of that medical image data that are acquired during the course of the same imaging sequence after acquisition of the medical image dataset.

For example, the reference person may thus observe the representation and the subsequent measurement dataset may be acquired during the course of repeated or continuous imaging, for example during the course of fluoroscopy and the control parameters may thus be dynamically adjusted within the imaging. For example, control parameters in respect of the acquisition, for example an X-ray intensity or image rate, may be dynamically adjusted in this connection. In addition, or alternatively, for example parameters that influence the subjective image impression, i.e. for example the “flavor” explained above, of the representation may be dynamically optimized.

The method may include the acquisition of the medical image dataset and/or the medical image data still to be acquired or also the entire repeated or continuous imaging. Alternatively, the acquisition of the medical image dataset and/or the medical image data may also take place outside of the method, however. The method may be carried out, for example, within the time interval after the end of the acquisition of the medical image dataset and before the acquisition of the medical image data still to be acquired. The described procedure may serve, for example, to optimize the quality measure explained above.

In addition or alternatively, it is possible to check for at least one object and/or at least one anatomical feature in each case whether a perception condition is fulfilled whose fulfilment depends on the respective measurement dataset and indicates a perception of the respective object or anatomical feature in the respective representation by the respective reference person, wherein the setpoint value or at least one of the setpoint values is ascertained as a function of the fulfilment of the respective perception condition.

A perception of particular objects or particular features by the respective reference person may indicate, for example during the course of an imaging sequence that is carried out to accompany the operation, that this object or anatomical features is currently particularly relevant. On the basis of this it is possible to establish approximately in real time and without prior knowledge or at least without detailed prior knowledge about the progress or planning of the operation whether it is currently relevant that the reference person may identify particular objects or anatomical features easily and reliably, for example while a microcatheter is being positioned.

If this is the case, i.e. for example with fulfilment of the perception condition, then, for example, higher image rates and/or a higher X-ray dose may be used to improve the imaging quality and thus further facilitate identification of relevant objects or features.

Otherwise, i.e. for example if the reference person averts their attention from the object or feature and the perception condition is thus no longer fulfilled, the control parameters may be adjusted in such a way, for example, that exposure to radiation or other stresses for the patient and, in addition or alternatively, also costs and/or the energy consumption of the imaging may be reduced, with it being possible to accept in this regard that the identification of the object or of the feature by the reference person is made more difficult.

If the identification of the object or feature or a different object or feature is relevant again at a subsequent instant, the reference person will focus on this identification and thus the perception of the respective object or feature is identified again and the imaging or the at least one control parameter may be adjusted accordingly.

The control parameter or a respective one of the control parameters may specify an X-ray dose irradiated onto an examination object for acquisition of the medical image data and/or imaging rate for acquisition of the medical image data. This may be expedient, for example, in the case of fluoroscopy or other imaging modalities with approximately real-time imaging, for example in the case of imaging that accompanies an operation.

In addition, or alternatively, setpoint values may also be ascertained for other control parameters used in the acquisition of the medical image data. In addition, or alternatively, setpoint values may be ascertained for control parameters, that relate to representing the medical image data or a processing result ascertained from the image data. This may be, for example, control parameters for a display facility, for example for setting an image brightness or a contrast. Alternatively, or in addition, setpoint values may be specified for control parameters in respect of a processing of the image data, for example parameters of an image reconstruction, an edge sharpening, an averaging over time and/or a noise suppression.

The control parameter or at least one of the control parameters, for which a setpoint value is specified, may control an enhancement of the medical image data with an item of semantic information and/or a segmentation of the medical image data or a processing result ascertained as a function of the medical image data, wherein the enhancement and/or segmentation is part of the processing of the medical image data.

For example, the perception condition explained above may be evaluated for this purpose in order to ascertain whether particular anatomical features and/or objects are identified in the representation by the reference person. It may be expedient, for example, if the measurement data also relates to at least regions of the brain that play a part in the semantic identification of image contents since in this case the brain of the reference persons may be co-used more or less as an identification algorithm or part of the identification algorithm.

As is already known for everyday articles, such as for aircraft, teddy bears, clocks and animals, measurement data in respect of the visual cortex of the brain may be merged with such items of semantic information in order to ascertain the position and/or size of the object or anatomical feature in the representation described by the semantic information. This may serve, for example, to add a classification or a label or the like to an anatomical feature or an object during the course of the enhancement of an item of semantic information, and/or to support segmentation of the medical image data or the processing result. For example, an initial segmentation may be specified by the control parameter and this may then be refined by known segmentation methods or the like.

For example, the medical image dataset may itself be part of the medical image data or form it. The method may thus also be used to segment image datasets while also using the brain of the reference person or to supplement with semantic information.

In addition or alternatively, a trained function may be trained that serves for processing of the medical image data or for use during the course of processing of the medical image data, wherein the control parameters are parameters of the trained function whose setpoint values are specified by machine learning during the course of supervised learning, wherein a plurality of training datasets is used for the supervised learning, that in each case include at least one of the measurement datasets or that depend in each case on at least one of the measurement datasets.

For example, items of semantic information in respect of at least one object mapped in the respective representation and/or anatomical feature may be ascertained during the course of the training or generation of the training datasets on the basis of the measurement data, and may be specified, for example, as a desired result of the trained function as part of the training dataset.

In general, a trained function maps cognitive functions which people associate with other human brains. By way of training on the basis of training data (machine learning), the trained function is capable of adjusting to new circumstances and detecting and extrapolating patterns.

Parameters of a trained function may be adjusted by way of training. For example, supervised learning, semi-supervised learning, unsupervised learning, reinforcement learning and/or active learning may be used. Furthermore, representation learning (also known as “feature learning”) may also be used. The parameters of the trained function may be adjusted, for example iteratively, by a plurality of training steps.

A trained function may include, for example, a neural net, a Support Vector Machine (SVM), a decision tree and/or a Bayesian network and/or the trained function may be based on k-means clustering, Q-Learning, genetic algorithms and/or allocation rules. For example, a neural network may be a deep neural network, a Convolutional Neural Network (CNN) or a deep CNN. Furthermore, the neural network may be an adversarial network, a deep adversarial network and/or a Generative Adversarial Network (GAN).

The trained function may be configured to carry out an enhancement of the medical image data with an item of semantic information and/or a segmentation of the medical image data. Items of semantic information extracted from the measurement dataset or items of segmentation information are thus, for example, not used, or at least not exclusively directly used, for segmentation or for labeling the medical image data. Instead, this information is used indirectly via the training of a trained algorithm. This may serve, for example, to transfer the knowledge and understanding of the reference person or, for example, a group of reference persons at least partially to the trained function.

Ascertaining the setpoint value as a function of the measurement dataset may include applying a function trained by machine learning to the measurement dataset and/or processing data ascertained from it. Possible embodiments of such trained functions and of their training have already been explained above. In addition, or as an alternative to the embodiments of the trained functions explained above, for example a transformer, specifically a vision transformer, may be used as the deep learning architecture as the trained function, that forms the trained function or a partial function of the trained function.

The algorithm trained by machine learning may serve, for example, to at least partially reconstruct or approximate the representation on the basis of the measurement dataset and/or obtain at least one item of semantic information in respect of an image content, for example a mapped object or anatomical feature. Implementation possibilities for this are already known from the articles cited in the introduction by Yu Takagi et al. and the article likewise cited in the introduction by Zijiao Chen et al. on the representation of viewed everyday articles or on their semantic identification or classification. It has been found that the approaches represented there in detail may be transferred by way of the use of suitable training data to medical imaging.

Suitable training datasets may be provided by experts, for example by way of manual classification of objects that may be seen in a respective representation or the underlying medical image dataset and/or anatomical features.

Training may alternatively or in addition also take place in that a representation observed by a respective reference person is repeatedly compared with a representation reconstructed from the measurement data for this reference person and the deviation is minimized during the course of training. This may take place, for example, by way of a backpropagation or also a generative adversarial network.

Embodiments provide a computer-implemented method for representing medical image data for an observer, wherein the acquisition of the medical image data and/or the processing of the medical image data for providing a processing result to be represented and/or representation of the medical image data and/or of the processing result are in each case parameterized by at least one control parameter, with the setpoint value used for the respective control parameter being specified by the computer-implemented method for ascertaining a respective setpoint value for at least one control parameter.

Embodiments provide a processing apparatus that is configured for carrying out the computer-implemented method for ascertaining a respective setpoint value for at least one control parameter and/or the computer-implemented method for representing medical image data for an observer.

The processing apparatus may be provided, for example, by way of suitable programming of a programmable data processing apparatus. Alternatively, a hard-wired processing apparatus, for example, may also be used, however.

The processing apparatus may be integrated, for example, in a medical imaging facility or also be configured separately from it. For example, the processing apparatus may be implemented as a workstation computer, server or also as a Cloud solution.

Embodiments provide a computer program, including program instructions that are configured to implement the computer-implemented method for ascertaining a respective setpoint value for at least one control parameter and/or the computer-implemented method for representing medical image data for an observer when they are executed on a data processing apparatus.

Embodiments further provide a data carrier that includes the computer program.

### BRIEF DESCRIPTION OF THE FIGURES

FIG. 1 depicts an embodiment of a processing apparatus and its use in the context of medical imaging.

FIG. 2-4 depict the interaction of an embodiment of the computer-implemented method for ascertaining a respective setpoint value for at least one control parameter with an embodiment of the method for representing medical image data.

FIG. 5-6 depict possible embodiments of trained functions that may be used in the context of the method.

### DETAILED DESCRIPTION

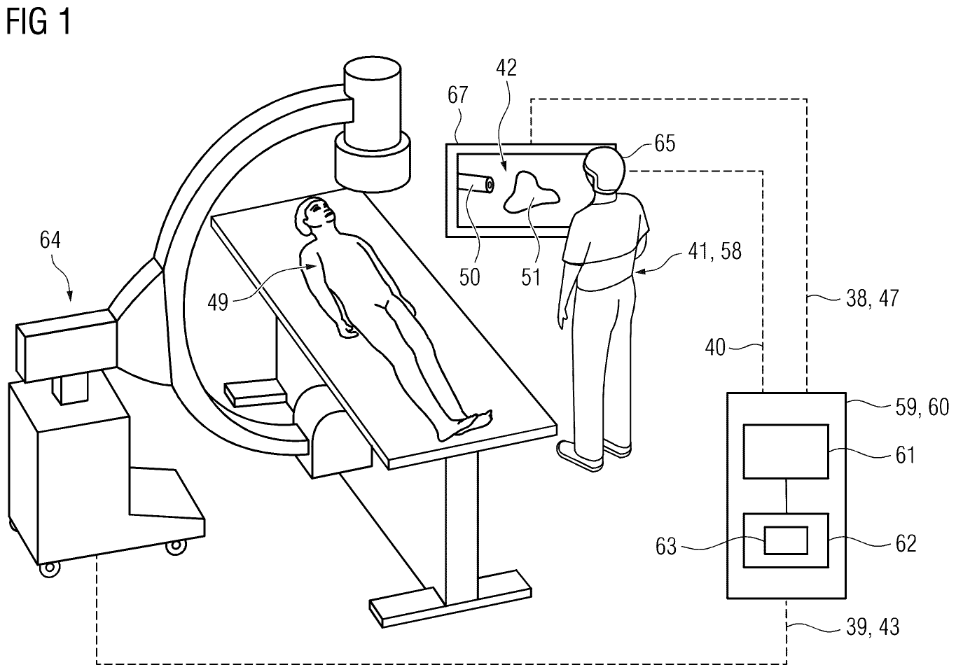

FIG. 1 depicts a medical imaging, with medical image data 39 or a processing result 47 ascertained from it being represented for an observer 58 by a processing apparatus 59.

Purely by way of example it is assumed that more or less continuous imaging with a relatively high imaging rate, for example fluoroscopy, is used for ascertaining the representation 42, with the medical image data 39 being directly provided by a medical imaging facility 64. The procedure described below may also be transferred to other methods for medical imaging, however, for example to any two-dimensional or three-dimensional and/or functional imaging procedures, for example on the basis of X-ray radiation, magnetic resonance, positron emission or the like. Processing may also include a reconstruction of three-dimensional image data. In addition, or as an alternative, it is also possible that the representation 42 is based on medical image data 39 that was clearly acquired before the explained processing and is read out, for example, from a database.

In medical imaging it is possible, as a rule, to parameterize the acquisition and/or processing and/or representation of the medical image data 39 by way of a large number of control parameters. In order to determine suitable setpoint values for at least some of these control parameters, the processing apparatus 59 is configured to evaluate the brain activity of at least one reference person 41 who observes the representation 42. In the example the reference person 41 is identical to the observer 58 but may also be a different person.

The processing apparatus 59 firstly actuates a display facility 67 for outputting a representation 42 of a medical image dataset 43 acquired in the example as part of the medical image data 39 or a processing result 47 of the image dataset 43. A control parameter 47 for controlling the display facility 67, for example for setting a brightness or a contrast, and/or control parameters, not represented in FIG. 1, in respect of the acquisition or processing of the image dataset 43 are initially set to specified reference values that have been strictly specified, for example, or have been determined as the setpoint values in a preceding iteration.

By way of a sensor 65, that is represented in an abstract manner in the example as a helmet worn by the reference person 41, a measurement dataset 40 is then acquired that relates to the brain activity of the reference person 41 while the person observes the representation 42. In order to provide the measurement dataset 40, the sensor 65 may carry out, for example, a functional imaging of the brain, for example based on magnetic resonance, and/or measure brain waves. In alternative embodiments it would also be possible to use a chip implanted in the brain of the reference person 41 or a different computer-brain interface for acquisition of the measurement dataset 40.

On the basis of this measurement dataset 40, a respective setpoint value may then be specified for at least one of the control parameters, that may then be used for the acquisition or processing or representation of medical image data acquired after the medical image dataset 43 or medical image data that is still to be acquired. This is expedient, since, as already explained in detail in the general part, using the brain activity or the measurement dataset 40 it is possible to identify, for example, whether or how easily the reference person 41 identifies an object 50 or an anatomical feature 51 in the representation 42 or whether the attention of the reference person 41 is even directed toward the representation 42 at all.

Some specific examples of a specification of setpoint values will be explained below. The individual examples combine in each case different developments of the procedure generally explained above, so individual features or implementation details of the following examples may also be omitted or features of the different explained examples may be combined.

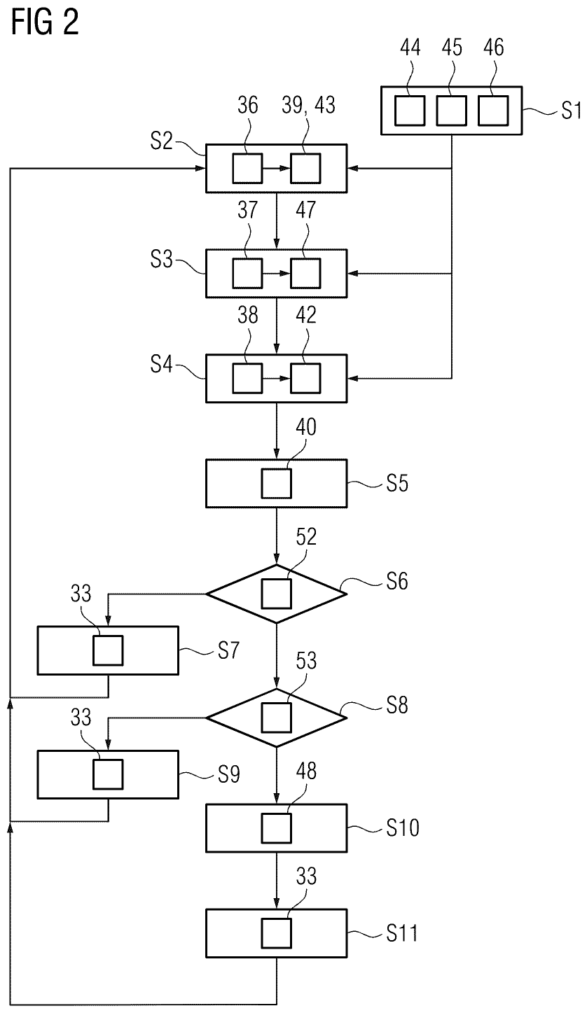

FIG. 2 depicts a flowchart of a method within which medical image data 39 is acquired and represented for an observer 58.

During the course of initialization, reference values 44, 45, 46 are firstly specified in step S1 for diverse control parameters 36, 37, 38 used during the course of the method. In the example the reference value 44 specifies the control parameter 36 that parameterizes acquisition of the medical image data 39. The reference value 45 specifies the control parameter 37 that parameterizes processing of the medical image data 39. The reference value 46 specifies the control parameter 38 that parameterizes representation of the medical image data 39.

In step S2, the medical image data 39 is acquired, for example by the imaging facility 64 shown in FIG. 1. In the example an imaging sequence is used in which medical image data 39 is repeatedly acquired on the same examination object 49, with one medical image dataset 43 respectively being acquired in the example in the respective iteration in step S2. The acquisition of the medical image data 39 or of the respective image dataset 43 is parameterized by the control parameter 36 that in the example specifies an X-ray dose irradiated during the course of the acquisition of the respective image dataset 43, for example by specifying a voltage and/or a current for an X-ray tube that is used and/or by adjusting the exposure time.

In step S3, the respective image dataset 39 is processed in order to provide a processing result 47. The processing is parameterized by the control parameter 37 that in the example specifies the extent of a noise suppression, for example by parameterization of a filter kernel used for this purpose.

In step S4, the processing result 47 and thus the medical image data 39 are represented for the observer 58. The representation is parameterized by the control parameter 38 that may control, for example, the display facility 67 in order to adjust a brightness and/or a contrast of the representation 42.

In the example the value of the control parameter 36 and thus the parameterization of the acquisition of the medical image data 43, that is acquired during subsequent iterations of the method, should be automatically adjusted to the needs of the observer 58. The observer thus serves as the reference person 41 for this adjustment.

For the sake of simplicity, the values of the control parameter 37, 38 in the example shown in FIG. 2 are not adjusted between the iterations. As already explained in the general part, however, or as will be represented in the further examples, these control parameters 37, 38 may also potentially be adjusted accordingly.

Since the following steps S5 to S 11 thus serve to specify a setpoint value for a control parameter, they may, independently of the previously explained steps, be regarded as a computer-implemented method for ascertaining a setpoint value for a control parameter.

In step S5, a measurement dataset 40 is acquired that is based on an acquisition by way of sensors of a respective brain activity of the reference person 41 while they are observing the representation 42. In the example the sensor 65 for acquisition of the measurement dataset is represented in an abstract manner as a helmet. Technically the brain activity may be acquired, for example, by a measurement of brain waves and/or functional imaging, for example by a functional magnetic resonance measurement.

If, as is indicated in FIG. 1, real-time imaging that accompanies an operation is used, and if, as in the example shown in FIG. 2, the setpoint value 33 is to be iteratively adjusted within the measuring sequence, then the sensor 65 should continue to enable sufficient freedom of movement of the reference person 41. If, by contrast, no movement of the reference person 41 is necessary, magnetic resonance scanners, for example, may also be used for more accurate acquisition of the brain activity, in which scanner the reference person 41 lies while the respective representation 42 is being displayed for them.

As already explained in the general part of the description, information may be obtained on the basis of the measurement dataset 40 by way of suitable algorithms, for example those trained by machine learning, which image contents the reference person 41 sees or identifies in the representation and how robustly and unambiguously this identification takes place. Supplementary implementation details may also be found in the articles cited in the introduction by Yu Takagi et al. and Zijiao Chen et al.

The evaluation of the measurement dataset 40 serves in the example to reduce the X-ray intensity irradiated onto the examination object 49 during the course of imaging if the reference person 41 has not directed their attention to the representation 42. For this purpose, in the example an anatomical feature 51 is selected in the mapped region of the examination object 49, for which feature it may be assumed that it may be easily identified over the entire admissible setting range of the control parameter 36 and thus the irradiated X-ray dose for the reference person 41 if the reference person 41 has directed their attention to the representation 42.

In step S6, it is then checked for this anatomical feature 51 whether a perception condition 52 is fulfilled whose fulfilment depends on the respective measurement dataset 40 and that indicates a perception of the anatomical feature 51 in the respective representation 42 by the reference person 41. For this, it may, for example, be checked whether an item of semantic information, extracted on the basis of the measurement dataset and that relates to the content of the viewed representation, identifies the anatomical feature 51.

If the perception condition 52 is not fulfilled in step S6 then this indicates that the reference person 41 is not currently directing their attention to the representation 42. Compared to other approaches for identifying attention, for example eye tracking, situations may also be identified in which the representation lies outside of the focal plane of the reference person 41 or the like.

Since the reference person 41 is not currently using the representation 42 in this case or is at best showing it very little attention, for example a progression of an operation accompanied by the imaging or a different sequence may be identified even without detailed knowledge such that, at least temporarily, the irradiated X-ray dose and thus the exposure of the examination object 49 may be reduced.

For this purpose, a setpoint value 33 may be specified in step S7 for the control parameter 36 that results in minimal exposure to radiation. To also enable an automatic increase in the X-ray dose and thus the image quality at a subsequent instant, in the example the setpoint value 33 is selected, however, such that the reference person 41 may continue to identify the anatomical feature 51 when they direct their attention to the representation 42, so in subsequent iterations the perception condition 52 is fulfilled as soon as the reference person 41 directs their attention to the representation 42 again.

If the perception condition 52 is fulfilled, by contrast, then a further perception condition 53 is checked in step S8 whose fulfilment indicates the perception of the object 50, for example of a microcatheter to be positioned, by the reference person 41. If the imaging serves, for example, to assist the reference person 41 in the positioning of the microcatheter, then it is essential that the reference person 41 may always identify it when their attention is directed toward the representation 42. Therefore, if the perception condition 53 is not fulfilled, in step S9 the setpoint value 33 is specified for the control parameter 36 in such a way that an optimum image quality and thus a robust identification of the object 50 by the reference person 41 is probable.

To avoid the use of unnecessarily high X-ray doses even when the reference person 41 has directed their attention to the representation 42 and thus in the example it must be possible to clearly identify the object 50, a quality measure 48 is ascertained for the representation 42 when the perception condition 53 is fulfilled in step S10. For this, it may, for example, be evaluated how unambiguously the semantic content of the representation 42 may be determined, i.e. for example how different an ascertained probability for the selected classification is from the probability for the next most probable classification, and/or how robustly the semantic content may be identified, for example to what extent the probability for the selected classification lies above a threshold value, only above which value does identification take place.

In step S11, the setpoint value 33 may then be ascertained in that an offset selected as a function of the quality measure 48 is added to the previously used setpoint value 33 for the control parameter 36 or in the first iteration, to the reference value 44, that offset may be positive or negative. The quality measure 48 may be regulated hereby to a specified value or in a specified interval, whereby, on the one hand, a robust ability for the reference person 41 to identify the object 50 may be ensured and, on the other hand, irradiation of an unnecessarily high X-ray dose onto the examination object 49 may be avoided.

The explained method is implemented in FIG. 1 by a processing apparatus 60 that is formed by a suitable programmed data processing apparatus 59. In the example the data processing facility 59 includes a processor 61 that may be formed, for example, by a microprocessor, a microcontroller, an FPGA, a graphics processor or the like. Stored in an associated memory 62 is a computer program 63 whose instructions the method implements. In addition, or alternatively, the methods still to be explained below or further methods explained in the general part and method features may also be implemented by using a suitable computer program 63.

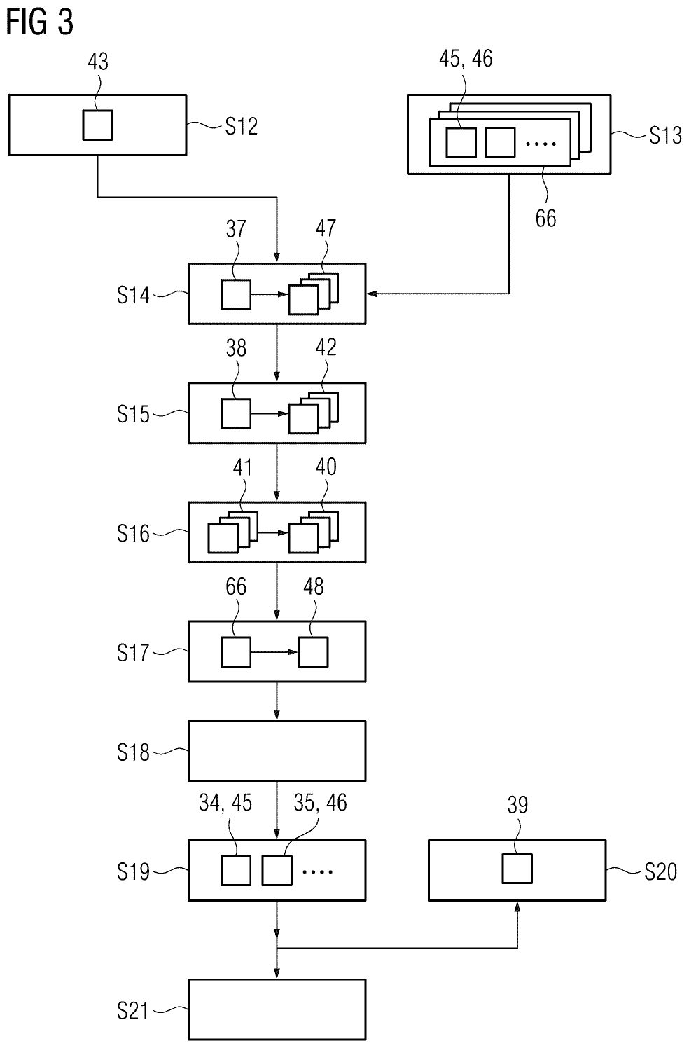

FIG. 3 depicts an alternative method for ascertaining a respective setpoint value 34, 35 for a respective control parameter 37, 38, with it being assumed in the example for the sake of simplicity that the control parameters 37, 38 should relate exclusively to the processing and representation of the medical image data 39. In the example, specifically a desired image impression 66, that may also be referred to as the “flavor”, should be selected for the image representation from a plurality of possibilities, that results in optimum identification of image contents. Reference values 45, 46 for the control parameters 37,38 are associated with each desired image impression 66 here, the use of which values results in the desired image impression 66.

In contrast to the previously discussed example, the selection of the setpoint values 34, 35 in FIG. 3 should not be optimized for a single reference person. Instead, a group study takes place in which instances of brain activity of a plurality of reference persons 41 are to be taken into account when observing representations 42 with different desired image impressions 66.

For this, at least one medical image dataset 43 is specified in step S12, for example by an acquisition of the image dataset 43 or by reading out the image dataset 43 from a database of previously acquired image datasets. For the sake of simplicity, it will be assumed in the following explanation that only a single image dataset 43 is being processed.

In step S13, one reference value 45 for the control parameter 37 and one reference value 46 for the control parameter 38 respectively is specified for each of the possible image impressions 66. For the sake of simplicity, it will be assumed that the processing and representation of the image dataset 43 is in each case parameterized by exactly one control parameter 37, 38. However, a plurality of control parameters may also be used for which a respective reference value may be specified as part of the desired image impression 66 or it is also possible to parameterize only the processing or only the representation by specifying the desired image impression.

In step S14, the image dataset 43 is then processed as a function of the control parameter 37 in order to generate an associated processing result 47 for each of the possible image impressions 66 and thus for each of the reference values 45. Apart from providing separate processing results 42 for separate reference values 45, the processing corresponds to the processing already explained in FIG. 2 with regard to step S3.

In step S15, different representations 42 of the image dataset 43 or of its processing results 47 are then output for each of the reference persons 41, with a plurality of representations 42 being output for each processing result 47 in that a different one of the reference values 46 respectively is used for the control parameter 38. As a result, a representation 42 is output to each of the reference persons 41 for each of the possible image impressions 66. The representation as a function of the control parameter 38 has already been explained in FIG. 2 with regard to step S4.

In step S16, one measurement dataset 40 respectively is acquired for each pair of a respective representation 42 and a respective reference person 41, which dataset relates to the brain activity of the respective reference person 41 while they are observing the respective representation 42. The acquisition of such measurement datasets 40 has already been explained in FIG. 2 with regard to step S5.

In step S17, an algorithm, in the example a trained function 66, is used to ascertain on the basis of a respective group of measurement datasets 40, that are based on an acquisition by way of sensors of the respective brain activity of different reference persons 41 when observing the same representation 42, a respective quality measure 48 for the respective representation 42.

In the simplest case individual quality measures for the respective measurement dataset 40 may be ascertained for this, as has already been explained in FIG. 2 with regard to step S10, and a mean or median may then be found or, for example, a weighted sum calculated in order to determine the shared quality measure 48 for the respective representation 42.

Alternatively, the algorithm or the trained function 66 may also be configured, however, to jointly process a plurality of measurement datasets, that relate to the observation of the same representation by different reference persons 41. Approaches to the use of a trained function for determining the quality measure 48 have already been discussed in the general part.

In the example, that representation is then selected in step S18 for which the highest value of the quality measure 48 results. In step S19, the reference parameters 45, 46 used to generate the selected representation 42 may then be directly provided as setpoint values 34, 35 for the control parameters 37, 38 for processing the medical image data 39 that is then to be processed. A suitable image impression 66 or “flavor” is thus automatically selected.

In an embodiment of the method, a plurality of the representations 42 could also be selected. For example, a subgroup of the representations 42 may be selected that includes the representation 42 with the highest quality value 48 and further representations 42 whose quality value 48 undershoots the highest quality value 48 at most by a specified threshold value. The setpoint values 34, 35 may then be ascertained, for example, as a mean or median or weighted sum of the respectively associated reference values 45, 46 of the representations of the subgroup.

It is also possible in addition or alternatively to carry out the described procedure on the basis of a plurality of image datasets 43 that are provided in step S12. In this case, for example all quality values 48, that are based on the same parameterization of the control parameters 37, 38, i.e. that are associated with the same possible image impression 66, may be added up to then select the suitable parameterization or the like on the basis of this sum.

The setpoint values 34,35 ascertained in step S19 may then be used to process and represent medical image data 39 provided in step S20, that may be, for example, a further image dataset, in step S21 in the same way as was explained above for the image dataset 43, with the control parameters 37, 38 being set to the setpoint values 34, 35. A type of presetting may thus be provided for the processing and representation of medical image data, which on the basis of the study in respect of the reference person 41 enables easy and robust identification of relevant image content probably for a large number of observers 58.

In the method shown in FIG. 3 the parameterization ascertained in step S19 may also be used to generate a representation of the image dataset 43, that is used as image data 39, or the representation selected in step S18 may be output for an observer 58. An optimum image impression for existing image data 39 may be selected hereby.

Instead of the parallel generation of a large number of representations 42 they may also be generated iteratively, similar to the manner explained in the method with regard to FIG. 2. For this the method explained with regard to FIG. 2 may be modified, for example, in such a way that an acquisition of the image dataset 43 or the image data 39 takes place only in the first iteration or is not part of the method. Instead of the specification of a setpoint value 33 for the control parameter 36 setpoint values 34, 35 may then be specified for the control parameters 37 and/or 38. This may serve, for example, to dynamically find an optimum representation for a particular reference person 41 for medical image data 39 that has already been acquired.

In principle it is also possible that procedures explained with regard to FIG. 3 are alternatively or additionally used to ascertain a setpoint value 33 for a control parameter 36 that parameterizes the acquisition of the medical image data 39. For example, a plurality of image datasets 43 for the same examination object 49, for example for a phantom, with different parameterization may be acquired for this in order to select the optimum parameterization.

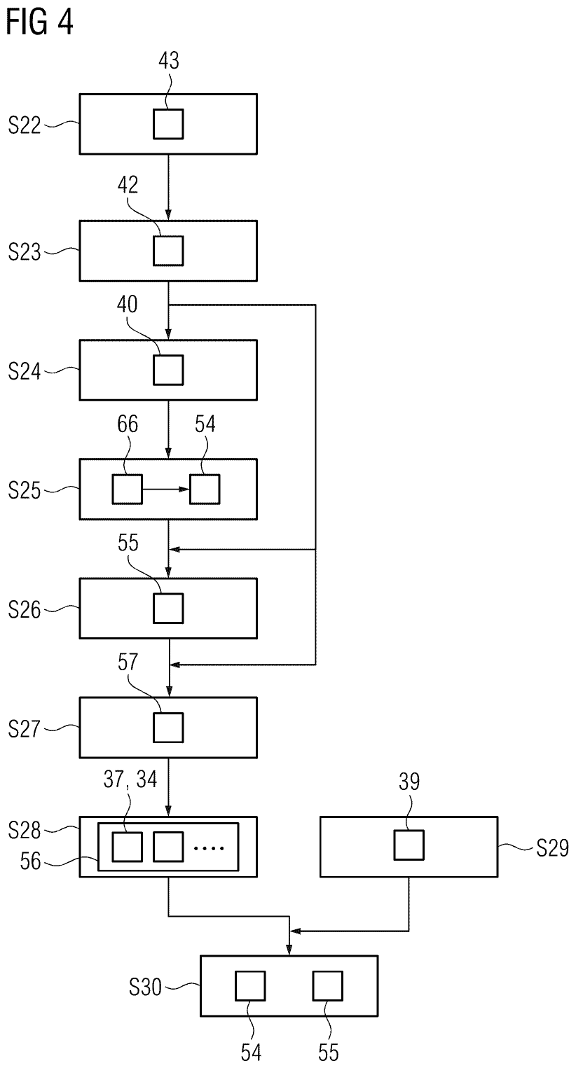

In addition, or as an alternative to the applications explained above, the acquisition of the brain activity of a reference person 41 during observation of a representation of an image dataset or of medical image data may also serve to enhance the medical image data with items of semantic information or to ascertain a segmentation. For example, in the representation 42 shown in FIG. 1 the object 50 and/or the anatomical feature 51 may be segmented and marked with an item of semantic information, i.e. for example with a label. One possible approach to this will be explained in more detail below with regard to FIG. 4.

In this connection direct segmentation and semantic supplementation of medical image data 39 will be discussed firstly with regard to steps S22 to S26, with this being used as the medical image dataset 43 to be represented. Following this it will then be explained with regard to steps S27 to S30 how this approach may be used to train a trained function 56 that may then also segment medical image data 39 different from the image dataset 43 or may supplement by way of items of semantic information.

In step S22, an image dataset 43 is firstly specified, for example acquired via the imaging facility 64 or is also taken from a database.

In step S23, a representation 42 of the image dataset 43 or a processing result of the image dataset 43 is output to the reference person 41 or, for example, a group of reference persons 41. Possibilities for generating a corresponding representation 42 have already been explained previously.

In step S24, a respective measurement dataset 40 is acquired that relates to the brain activity of the respective reference person 41 while they observe the representation 42.

Items of semantic information in respect of articles identified or at least seen by the respective reference person 41 in the representation 42 are then extracted by an algorithm, that in the example is a trained function 66, in step S25 on the basis of the measurement dataset 40 or the measurement datasets 40.

As has already been represented in detail in the general part or in the professional articles cited above, items of semantic information may be directly extracted on the basis of the measured brain activity or an image may be reconstructed, for example on the basis of the respective measurement dataset 40, on the basis of which reconstruction represented articles may then be identified or classified.

After a successful identification of the represented articles for at least some of the measurement datasets 40 it may thus be ascertained for the representation 42 in FIG. 1, for example as the semantic information 54, that a microcatheter was identified or at least seen as the article 50 and the anatomical feature 51 by the reference persons 41 or at least some of the reference persons 41.

In step S26, a segmentation 55 for the representation 42 or, generally, for the image dataset 43 may then be determined. A known segmentation algorithm may be used here that may also use the semantic information 54, however, and thus likewise depends on the at least one measurement dataset 40. As has likewise already been explained in the general part, however, identified semantic features may also be localized in the representation 42 using the measurement dataset 40, so the measurement dataset 40 may already specify a rough segmentation that may then still be refined or the like by using segmentation approaches that work iteratively.

An improvement in the robustness of the identified semantic information 54 or the achieved segmentation 55 may be achieved in that, as explained above, measurement datasets 40 are acquired for a plurality of reference persons 41. A plurality of different representations 42 may also be generated in that, as already explained above, control parameters 37, 38 for the processing and/or representation of the image dataset 43 are varied to be able to evaluate a higher number of different measurement datasets 40 overall.

If the semantic information 54 and/or the segmentation 55 is considered directly as control parameters 37 for the processing of the medical image dataset 43, then this may be identified with the medical image data 39 and a method for ascertaining setpoint values for control parameters 37, that serve to control processing of medical image data 39, is thus already concluded with step S25 or step S26.

Steps S22 to S26 are used, however, to provide in step S27 training datasets for trained of a trained function 56 by machine learning. The segmentation 55 explained above and/or the supplementation of semantic information 54 for a large number of different image datasets 43 or representations 42 resulting from them may be carried out for this, whereby one training dataset 57 respectively may be provided that includes as input data the medical image dataset 43 or, in the example, the representation 42 and as the desired result, that is used during the course of training, the semantic information 54 and/or the segmentation 55.

If a sufficient number of these training datasets 57 is provided, then in step S28 the trained function 56 may be parameterized or trained by conventional approaches to supervised learning, for example by a backpropagation, in such a way that it may carry out the ascertainment of the semantic information 54 and the segmentation 55 for a large number of medical image data 39. The control parameters 37 for processing the medical image data 39, for which the setpoint values 34 are specified, are in this case the parameters of the trained function 56, that are learned during the course of machine learning, for example node weights in a neural net.

After training of the trained function 56 the medical image data 39 to be segmented or to be enhanced with items of semantic information may be provided in step S29, whereby the trained function 56 may be applied in step S30 to this medical image data 39 or a representation generated from this image data 39 in order to determine the semantic information 54 and the segmentation 55.

As has been explained above, the fundamental approach to using measurement data, that is based on an acquisition by way of sensors of a respective brain activity of a respective reference person, who is observing a representation of a medical image dataset, for parameterization of the acquisition and/or processing and/or representation of medical image data, may thus be used in a wide variety of ways to solve diverse relevant problems in medical imaging. As may be found, for example, in the general part of the description, neither the basic idea nor possible embodiments that develop it are limited to the specifically cited embodiment and the features of the embodiments that have been explained may be combined in a wide variety of ways.

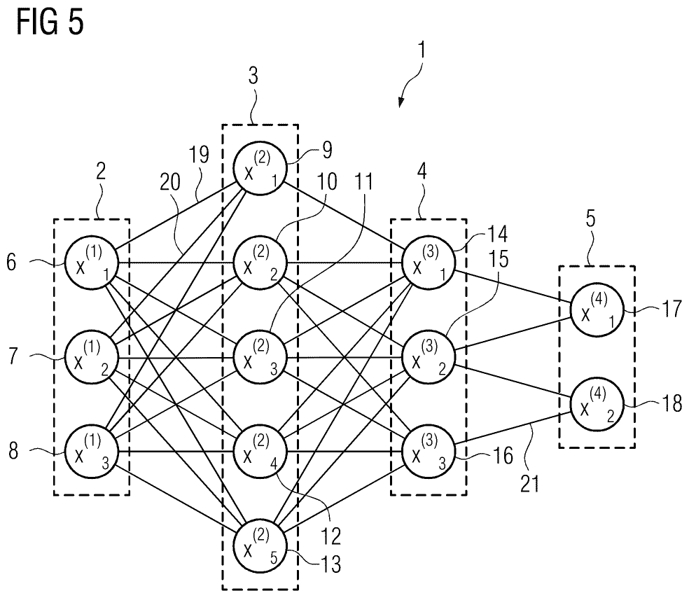

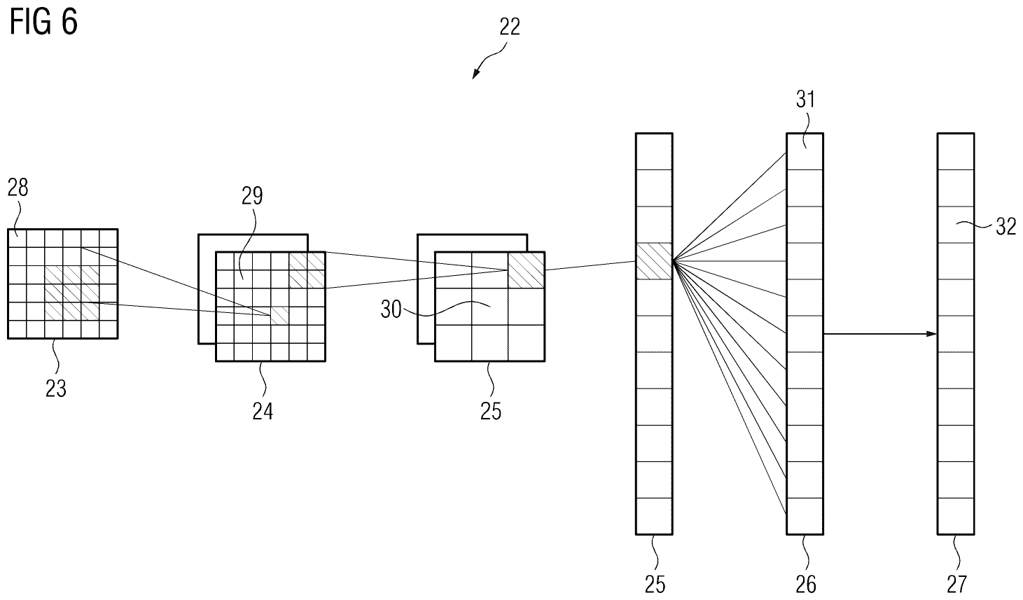

FIGS. 5 and 6 illustration examples of trained functions that may be used in the explained method, for example as trained function 56 and/or 66.

FIG. 5 depicts an embodiment of an artificial neural net 1. English expressions for the artificial neural net 1 are “artificial neural network”, “neural network”, “artificial neural net” or “neural net”.

The artificial neural network 1 includes nodes 6 to 18 (nodes) and edges 19 to 21 (edges), with each edge 19 to 21 being a directed connection from a first node 6 to 18 to a second node 6 to 18. In general, the first node 6 to 18 and the second node 6 to 18 are different nodes 6 to 18, but it is also conceivable for the first node 6 to 18 and the second node 6 to 18 to be identical. For example, in FIG. 5 the edge 19 is a directed connection from the node 6 to the node 9 and the edge 21 is a directed connection from the node 16 to the node 18. An edge 19 to 21 from a first node 6 to 18 to a second node 6 to 18 is referred to as the ingoing edge for the second node 6 to 18 and as the outgoing edge for the first node 6 to 18.

In this embodiment, the nodes 6 to 18 of the artificial neural net 1 may be arranged in layers 2 to 5, it being possible for the layers to have an intrinsic order that is introduced between the nodes 6 to 18 by the edges 19 to 21. For example, edges 19 to 21 may be provided only between adjacent layers of nodes 6 to 18. In the represented embodiment, there exists an input layer 2 that has solely the nodes 6, 7, 8, in each case without ingoing edge. The output layer 5 includes only the nodes 17, 18, in each case without outgoing edge, with hidden layers 3 and 4 being located between the input layer 2 and the output layer 5, moreover. In the general case the number of hidden layers 3, 4 may be arbitrarily selected. The number of nodes 6, 7, 8 of the input layer 2 customarily corresponds to the number of input values in the neural network 1, and the number of nodes 17, 18 in the output layer 5 customarily corresponds to the number of output values of the neural network 1.

For example, a (real) number may be associated with the nodes 6 to 18 of the neural network 1. In this case x⁽ⁿ⁾ᵢ denotes the value of the i^{th} node 6 to 18 of the n^{th} layer 2 to 5. The values of the nodes 6, 7, 8 of the input layer 2 are equivalent to the input values of the neural network 1, while the values of the nodes 17, 18 of the output layer 5 are equivalent to the output values of the neural network 1. Furthermore, a weight in the form of a real number may be associated with each edge 19, 20, 21. For example, the weight is a real number in the interval [−1, 1] or in the interval [0, 1,]. In this case w^{(m,n)}_{i,j} denotes the weight of the edge between the i^{th} nodes 6 to 18 of the m^{th} layer 2 to 5 and the j^{th} nodes 6 to 18 of the n^{th} layer 2 to 5. Further, the abbreviation w is defined for the weight w_{i,j}^{(n,n+1)}.

In order to calculate output values of the neural net 1, the input values are propagated by the neural net 1. For example, the values of the nodes 6 to 18 of the (n+1)^{th} layer 2 to 5 may be calculated on the basis of the values of the nodes 6 to 18 of the n^{th} layer 2 to 5 by

$${x}_{j}^{( n + 1 )} = f ( {\sum }_{i} {x}_{i}^{( n )} \cdot {w}_{i , j}^{( n )} ) .$$

In this case f is a transfer function that may also be referred to as an activation function. Known transfer functions are step functions, sigmoid functions (for example the logistic function, the generalized logistic function, the hyperbolic tangent, the arc tangent, the error function, the smoothstep function or rectifier functions (rectifier). The transfer function is substantially used for standardization purposes.

For example, the values are propagated layer-wise by the neural net 1, with values of the input layer 2 being given by the input data of the neural net 1. Values of the first hidden layer 3 may be calculated on the basis of the values of the input layer 2 of the neural net 1, values of the second hidden layer 4 may be calculated on the basis of the values in the first hidden layer 3, etc.

To be able to define the values w for the edges 19 to 21 the neural net 1 has to be trained using training data. For example, training data includes training input data and training output data that is referred to below as ti. For a training step the neural network 1 is applied to the training input data in order to ascertain calculated output data. For example, the training output data and the calculated output data include a number of values, with the number being determined as the number of nodes 17, 18 of the output layer 5.

For example, a comparison between the calculated output data and the training output data is used to recursively adjust the weights within the neural net 1 (backpropagation algorithm). For example, the weights may be changed according to

$${w}_{i , j}^{{′}^{( n )}} = {w}_{i , j}^{( n )} - γ \cdot {δ}_{j}^{( n )} \cdot {x}_{i}^{( n )}$$

where y is a learning rate and the numbers δⱼ⁽ⁿ⁾ may be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

on the basis of δⱼ⁽ⁿ⁺¹⁾ if the (n+1)^{th} layer is not the output layer 5 is, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)^{th} layer is the output layer 5, where f is the first derivation of the activation function and yⱼ⁽ⁿ⁺¹⁾ is the comparison training value for the j^{th} node 17, 18 of the output layer 5.

An example of a convolutional neural network (CNN) will also be given below with regard to FIG. 6. It should be noted in this connection that the expression “layer” is used there slightly differently than for conventional neural nets. For a conventional neural net, the expression “layer” refers only to the set of nodes that forms a layer, therefore a particular generation of nodes. For a convolutional neural network, the expression “layer” is often used as an object that actively changes data, in other words as a set of nodes of the same generation and either the set of ingoing or outgoing edges.

FIG. 6 depicts an embodiment of a convolutional neural network 22. In the represented embodiment, the convolutional neural network 22 includes an input layer 23, a convolutional layer 24, a pooling layer 25, a fully connected layer 26 and an output layer 27. In alternative embodiments the convolutional neural network 22 may include a plurality of convolutional layers 24, a plurality of pooling layers 25 and a plurality of fully connected layers 26, as well as other types of layers. The order of the layers may be arbitrarily selected, with fully connected layers 26 conventionally forming the last layers before the output layer 27.

For example, within a convolutional neural network 22 the nodes 28 to 32 of one of the layers 23 to 27 may be understood as being arranged in a d-dimensional matrix or as a d-dimensional image. For example, in the two-dimensional case, the value of a node 28 to 32 with the indices i, j in the n^{th} layer 23 to 27 may be referred to as x⁽ⁿ⁾[i,j]. It should be noted that the arrangement of the nodes 28 to 31 of a layer 23 to 27 have no effect on the calculations within the convolutional neural network 22 as such since these effects are given solely by the structure and the weights of the edges.

A convolutional layer 24 is characterized for example in that the structure and the weights of the ingoing edges form a convolutional operation based on a particular number of kernels. For example, the structure and the weights of the ingoing edges may be selected such that the values xₖ^{(k)} of the nodes 29 of the convolutional layer 24 are ascertained as a convolution xₖ⁽ⁿ⁾=Kₖ*x⁽ⁿ⁻¹⁾ on the basis of the values x⁽ⁿ⁻¹⁾ of the nodes 28 of the preceding layer 23, it being possible to define the convolution * in the two-dimensional case as

$${x}_{k}^{( n )} [ i , j ] = ( {K}_{k} * {x}^{( n - 1 )} ) [ i , j ] = {\sum }_{i ′} {\sum }_{j ′} {K}_{k} [ {i}^{′} , {j}^{′} ] \cdot {x}^{( n - 1 )} [ i - {i}^{′} , j - {j}^{′} ] .$$

Here the k^{th} kernel Kₖ is a d-dimensional matrix, in this embodiment a two-dimensional matrix, that is conventionally small compared to the number of nodes 28 to 32, for example a 3×3 matrix or a 5×5 matrix. For example, this implies that the weights of the ingoing edges are not independent but are selected such that they generate the above convolutional equation. In the example for a kernel that forms a 3×3 matrix, there exist only nine independent weights (with each entry in the kernel matrix corresponding to an independent weight), regardless of the number of nodes 28 to 32 in the corresponding layer 23 to 27. For example, for a convolutional layer 24 the number of nodes 29 in the convolutional layer 24 is equivalent to the number of nodes 28 in the preceding layer 23 multiplied by the number of convolutional kernels.

If the nodes 28 in the preceding layer 23 are arranged as a d-dimensional matrix, use of the plurality of kernels may be understood as an addition of a further dimension, that is also referred to as a depth dimension, so the nodes 29 of the convolutional layer 24 are arranged as a (d+1)-dimensional matrix. If the nodes 28 of the preceding layer 23 are already arranged as a (d+1)-dimensional matrix with a depth dimension, the use of a plurality of convolutional kernels may be understood as an expansion along the depth dimension, so the nodes 29 of the convolutional layer 24 are arranged similarly to a (d+1)-dimensional matrix, with the size of the (d+1)-dimensional matrix in the depth dimension being greater than in the preceding layer 23 by the factor formed by the number of kernels.

The advantage of the use of convolutional layers 24 is that the spatially local correlation of the input data may be utilized in that a local connection pattern between nodes of adjacent layers is created, for example in that each node has connections only for a small region of the nodes of the preceding layer.

In the illustrated embodiment, the input layer 23 includes thirty six nodes 28 that are arranged as a kernel 6×6 matrix. The convolutional layer 24 includes seventy two nodes 29 that are arranged as two kernel 6×6 matrices, with each of the two matrices being the result of a convolution of the values of the input layer 23 with a convolutional kernel. Similarly, the nodes 29 of the convolutional layer 24 may be understood as being arranged in a three-dimensional 6×6×2 matrix, with the last-mentioned dimension being the depth dimension.

A pooling layer 25 is characterized in that the structure and the weights of the ingoing edges and the activation function of their nodes 30 define a pooling operation based on a non-linear pooling function f. For example, in the two-dimensional case, the values x⁽ⁿ⁾ of the nodes 30 of the pooling layer 25 may be calculated on the basis of the values x⁽ⁿ⁾ of the nodes 29 of the preceding layer 24 as

$${x}^{( n )} [ i , j ] = f ( {x}^{( n - 1 )} [ {id}_{1} , {jd}_{2} ] , … , {x}^{( n - 1 )} [ {id}_{1} + {d}_{1} - 1 , {jd}_{2} + {d}_{2} - 1 ] )$$

In other words, the number of nodes 29, 30 may be reduced by the use of a pooling layer 25 in that a number of d₁×d₂ adjacent nodes 29 in the preceding layer 24 are replaced by an individual node 30 that is calculated as a function of the values of the number of adjacent nodes 29. For example, the pooling function f may be a maximum function, an average or the L2 standard. For example, the weights of the ingoing edges may be defined for a pooling layer 25 and not be modified by training.

The advantage of using a pooling layer 25 is that the number of nodes 29, 30 and the number of parameters is reduced. This results in a reduction in the necessary calculation quantity within the convolutional neural network 22 and thus in control of the overfitting.

In the represented embodiment, the pooling layer 25 is a max pooling layer in which four adjacent nodes are replaced with just a single node whose value is formed by the maximum of the values of the four adjacent nodes. The max pooling is applied to each d-dimensional matrix of the previous layer; in this embodiment the max pooling is applied to each of the two two-dimensional matrices, so the number of nodes reduces from seventy two to eighteen.

A fully connected layer 26 is characterized in that a large number, for example all, edges between the nodes 30 of the previous layer 25 and the nodes 31 of the fully connected layer 26 are present, it being possible to individually adjust the weight of each of the edges. In this embodiment, the nodes 30 of the preceding layer 25 and the fully connected layer 26 are shown as two-dimensional matrices as well as non-contiguous nodes (represented as a row of nodes, with the number of nodes having been reduced for improved representation). In this embodiment, the number of nodes 31 in the fully connected layer 26 is equal to the number of nodes 30 in the preceding layer 25. The number of nodes 30, 31 may be different in alternative embodiments.

Furthermore, the values of the nodes 32 of the output layer 27 are determined in this embodiment in that the softmax function is applied to the values of the nodes 31 of the preceding layer 26. By applying the softmax function the sum of the values of all nodes 32 of the output layer 27 is one and all values of all nodes 32 of the output layer are real numbers between 0 and 1. If the convolutional neural network 22 is used for the classification of input data, for example the values of the output layer 27 may be interpreted as a probability for the input data falling into one of the different categories.

A convolutional neural network 22 may similarly have a ReLU layer, with ReLU standing as an acronym for “rectified linear units”. For example, the number of nodes and the structure of the nodes within a ReLU layer is equivalent to the number of nodes and the structures of the nodes of the preceding layer. The value of each node in the ReLU layer may be calculated, for example, by applying a rectifier function to the value of the corresponding node of the preceding layer. Examples of rectifier functions are f(x)=max(0,x), the hyperbolic tangent or the sigmoid function.

Convolutional neural networks 22 may be trained on the basis, for example, of the backpropagation algorithm. To avoid an overfitting, methods of regularization may be used, for example dropout of individual nodes 28 to 32, stochastic pooling, use of artificial data, weight decay based on the L1 or the L2 standard or maximum standard limitations.

It is to be understood that the elements and features recited in the appended claims may be combined in different ways to produce new claims that likewise fall within the scope of the present embodiments. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present embodiments have been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for ascertaining a setpoint value for at least one control parameter that serves to control at least one of an acquisition, processing, or a respective representation of medical image data, the method comprising:
obtaining at least one measurement dataset that is based on the acquisition by way of sensors of a brain activity of a reference person, while the reference person observes at least one representation, wherein the representation is based on a medical image dataset; and
ascertaining the setpoint value as a function of the at least one measurement dataset.

2. The computer-implemented method of claim 1, wherein the at least one representation depends on a reference value associated with the representation for the at least one control parameter, wherein the setpoint value for the at least one control parameter is also ascertained as a function of the reference value for the at least one control parameter that is associated with the representation.

3. The computer-implemented method of claim 2, wherein the representation, the acquisition of the medical image dataset and/or the processing of the medical image dataset for providing a processing result to be represented and/or representing the medical image dataset and/or the processing result, depends on the reference value associated with the representation for the at least one control parameter.

4. The computer-implemented method of claim 1, wherein on the basis of the measurement dataset or on the basis of a group of measurement datasets that are based on the acquisition by way of sensors of brain activity of different reference persons when a same representation is observed, a quality measure is ascertained for the representation, wherein the setpoint value is ascertained as a function of the quality measure.

5. The computer-implemented method of claim 4, wherein the at least one control parameter serves to control the processing and/or the representation of the medical image data, wherein on the basis of the medical image dataset in each case, a plurality of mutually different representations is generated in that processing and a representation of the medical image dataset take place in a same way as for the medical image data, wherein for providing the representation a reference value associated with the representation is used for specifying the at least one control parameter, wherein the mutually different representations differ from each other at least in respect of the reference value for the at least one control parameter, wherein as a function of the quality measure ascertained for the representation one of the representations or a subgroup of the representations that does not incorporate all representations is selected, according to which the setpoint value is specified for the at least one control parameter as a function of the reference value associated with a selected representation or of the reference value associated with the selected representation.

6. The computer-implemented method of claim 1, wherein the medical image dataset, on which the representation is based, is a medical image dataset ascertained during an imaging sequence, wherein the imaging sequence comprises repeated or continuous acquiring of medical image data on a same examination object, wherein the setpoint value serves to control the acquisition and/or the processing and/or the representation of at least parts of that medical image data that is acquired during the imaging sequence after acquisition of the medical image dataset.

7. The computer-implemented method of claim 1, wherein for at least one object and/or at least one anatomical feature it is in each case checked whether a perception condition is fulfilled whose fulfilment depends on the measurement dataset and indicates a perception of the at least one object or at least one anatomical feature in the representation by the reference person, wherein the setpoint value is ascertained as a function of the fulfilment of the perception condition.

8. The computer-implemented method of claim 1, wherein the at least one control specifies an X-ray dose irradiated onto an examination object for acquisition of the medical image data and/or imaging rate, for acquisition of the medical image data.

9. The computer-implemented method of claim 1, wherein the at least one control parameter for which a setpoint value is specified controls an enhancement of the medical image data with an item of semantic information and/or a segmentation of the medical image data or a processing result ascertained as a function of the medical image data, wherein the enhancement and/or segmentation is part of the processing of the medical image data.

10. The computer-implemented method of claim 1, wherein a trained function is trained that serves for processing the medical image data or for use during the processing of the medical image data, wherein the at least one control parameter is a parameter of the trained function whose setpoint values are specified by machine learning during the supervised learning, wherein a plurality of training datasets is used for the supervised learning, that in each case comprise at least one of the measurement datasets or which in each case depend on at least one of the measurement datasets.

11. The computer-implemented method of claim 1, wherein the trained function is configured to carry out an enhancement of the medical image data with an item of semantic information and/or a segmentation of the medical image data.

12. The computer-implemented method of claim 1, wherein ascertaining the setpoint value as a function of the measurement dataset comprises applying a function trained by machine learning to the measurement dataset and/or processing data ascertained from the measurement dataset.

13. The computer-implemented method of claim 1, wherein the acquisition of the medical image data and/or the processing of the medical image data for providing a processing result to be represented and/or representing the medical image data and/or the processing result are in each case parameterized by the at least one control parameter, wherein the setpoint value used for the at least one control parameter.

14. A non-transitory computer implemented storage medium that stores machine-readable instructions for ascertaining a setpoint value for at least one control parameter that serves to control an acquisition and/or processing and/or a representation of medical image data, the machine-readable instructions executable by at least one processor, the machine-readable instructions comprising:
obtaining at least one measurement dataset that is based on an acquisition by way of sensors of a brain activity of a reference person, while the reference person observes at least one representation, wherein a representation is based on a medical image dataset; and
ascertaining the setpoint value as a function of the measurement dataset.
